(19) United States
(12) Patent Application Publication
(10) Pub. No.: US 2024/0148240 A1
(43) Pub. Date: May 9, 2024

(54) ENDOSCOPE SYSTEM AND INTERFACE ADAPTER

(71) Applicant: FUJIFILM Corporation, Tokyo (JP)

(72) Inventor: Takeaki UCHIDA, Ashigarakami-gun (JP)

(73) Assignee: FUJIFILM Corporation, Tokyo (JP)

(21) Appl. No.: 18/413,038

(22) Filed: Jan. 16, 2024

Related U.S. Application Data
(63) Continuation of application No. PCT/JP2022/026126, filed on Jun. 29, 2022.

(30) Foreign Application Priority Data
Aug. 20, 2021 (JP) 2021-134912

(51) Int. Cl. A61B 1/05; A61B 1/00; A61B 1/06

(52) U.S. Cl. CPC A61B 1/05; A61B 1/00027; A61B 1/00045; A61B 1/0655

(57) ABSTRACT

An endoscope system includes: a scope that has an imaging sensor; a frame that houses a portable information terminal; and an interface adapter that converts an imaging signal obtained by the imaging sensor into image data displayable by the portable information terminal and sends the image data to the portable information terminal, and the interface adapter is fixed to the frame.

### CROSS REFERENCE TO RELATED APPLICATION

This is a continuation of International Application No. PCT/JP2022/026126 filed on Jun. 29, 2022, and claims priority from Japanese Patent Application No. 2021-134912 filed on Aug. 20, 2021, the entire disclosures of which are incorporated herein by reference.

### BACKGROUND OF THE INVENTION

### 1. Field of the Invention

The present invention relates to an endoscope system and an interface adapter.

### 2. Description of the Related Art

In recent years, a disposable endoscope that can be used hygienically safely by single time use has been attracting attention. JP2012-511357A describes an endoscope that is configured such that an image signal is transmitted from a camera of the endoscope to a control operating unit and the image signal is sent from the control operating unit to a video display via an electric wire. JP2020-18876A describes a laparoscope system that is configured such that a laparoscope communicates with a dongle and the dongle sends image data to a television display.

### SUMMARY OF THE INVENTION

It is, however, not possible in the aforementioned related art to, while enabling an image based on an imaging signal obtained by a scope to be displayed by a general-purpose portable information terminal, facilitate handling for operating the scope with the image being observed.

For example, in the configuration disclosed in JP2012-511357A, a control module that has a video display is a dedicated terminal compatible with the endoscope in order to process and display an image signal from a camera of the endoscope. Replacing and updating of such a dedicated terminal are difficult compared with general-purpose portable information terminals such as a tablet terminal.

In the configuration disclosed in JP2020-18876A, handling for operating the laparoscope while observing an image on a television display is difficult since the dongle connected by a cable to each of the laparoscope and the television display is present between the laparoscope and the television display.

Meanwhile, JP2020-18876A also describes that a mobile computing device, such as a tablet or a cellular phone, is used as an alternative to the dongle. In this case, however, a relay device having a processing circuit that converts an image signal from a camera of, for example, an endoscope into image data displayable by a general-purpose mobile computing device is required to be provided between the laparoscope and the mobile computing device, and there is still a problem that handling is difficult. Alternatively, this processing circuit may be considered to be provided in the laparoscope. In this case, however, manufacturing costs of the laparoscope increases, which makes utilization of the laparoscope in single time use difficult.

The present invention has been made in consideration of the aforementioned circumstances, and an object of the present invention is to provide an endoscope system and an interface adapter that can facilitate handling while enabling an image based on an imaging signal obtained by a scope to be displayed by a general-purpose portable information terminal.

An endoscope system according to one aspect of the present invention includes a scope that has an imaging sensor; a frame that houses a portable information terminal; and an interface adapter that converts an imaging signal obtained by the imaging sensor into image data displayable by the portable information terminal and sends the image data to the portable information terminal. The interface adapter is fixed to the frame.

An interface adapter according to one aspect of the present invention is an interface adapter of an endoscope system, the endoscope system including a scope that has an imaging sensor, and a frame that houses a portable information terminal having a display. The interface adapter includes an image processor that converts an imaging signal obtained by the imaging sensor into image data displayable by the display and sends the image data to the portable information terminal. The interface adapter is attachable to the frame.

The present invention can provide an endoscope system and an interface adapter that can facilitate handling while enabling an image based on an imaging signal obtained by a scope to be displayed by a general-purpose portable information terminal.

### BRIEF DESCRIPTION OF THE DRAWINGS

FIG. 1 is a diagram illustrating one example of an endoscope system that is one embodiment of the present invention;

FIG. 2 is a first diagram illustrating one example of an interface adapter;

FIG. 3 is a second diagram illustrating one example of the interface adapter;

FIG. 4 is a first diagram illustrating one example of a state of the interface adapter and a portable information terminal before being housed in a frame;

FIG. 5 is a second diagram illustrating one example of a state of the interface adapter and the portable information terminal before being housed in the frame;

FIG. 6 is a first diagram illustrating one example of a state of the interface adapter and the portable information terminal that are attached to a rear case;

FIG. 7 is a second diagram illustrating one example of a state of the interface adapter and the portable information terminal that are attached to the rear case;

FIG. 8 is a first diagram illustrating one example of a state of a front cover attached to the rear case;

FIG. 9 is a second diagram illustrating one example of a state of the front cover attached to the rear case;

FIG. 10 is a diagram illustrating one example of internal configurations of a scope and the interface adapter;

FIG. 11 is a diagram illustrating one example of a hardware configuration of the portable information terminal;

FIG. 12 is a diagram illustrating one example of an isolation portion included in the interface adapter;

FIG. 13 is a diagram illustrating one example of a power supply unit included in the interface adapter; and

FIG. 14 is a diagram illustrating one example of a power supply unit that uses electric power from the portable information terminal.

### DESCRIPTION OF THE PREFERRED EMBODIMENTS

Hereinafter, an embodiment of the present invention will be described with reference to the drawings.

### Endoscope System 100 as One Embodiment of Present Invention

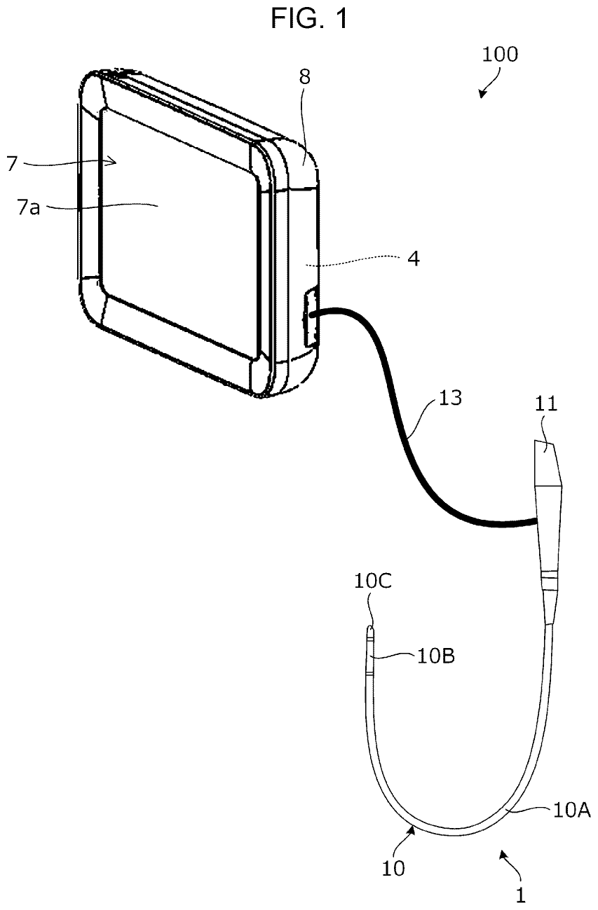

FIG. 1 illustrates one example of an endoscope system 100 as one embodiment of the present invention. The endoscope system 100 includes a scope 1, an interface adapter 4 connected to the scope 1, and a frame 8 that houses a portable information terminal 7 connected to the interface adapter 4. Note that, in FIG. 1, the interface adapter 4 is present on the back side of the portable information terminal 7 and covered by the frame 8 and thus is invisible. The endoscope system 100 may further include the portable information terminal 7.

The scope 1 is an endoscope that includes an insertion part 10 that is a tubular member extending in one direction and that is to be inserted into a subject, and an operation part 11 that is provided at a proximal end portion of the insertion part 10 and to be used to perform various operations of the scope 1. The operation part 11 includes, for example, an angle knob that is operated to turn to bend the insertion part 10. In addition, the operation part 11 may include an operating member and the like for performing an observation-mode switching operation, an imaging recording operation, a forcep operation, an air supply/water supply operation, a suction operation, and the like of the scope 1.

The scope 1 is connected to the interface adapter 4 via a communication cable 13. In addition, the scope 1 is attachable and detachable with respect to the interface adapter 4 via the communication cable 13 and can be used a single time (that is, disposable). For example, the communication cable 13 may be attachable and detachable with respect to the interface adapter 4, and the scope 1 may be attachable and detachable with respect to the communication cable 13.

Note that, although not illustrated in FIG. 1, the inside of the operation part 11 and the insertion part 10 may be provided with a forcep hole, into which a forcep for collecting a biological tissue such as a cell or a polyp is to be inserted, and various channels such as channels for air supply and water supply and a channel for suction.

The insertion part 10 is constituted by a flexible soft portion 10A, a bending portion 10B provided at a distal end of the soft portion 10A, and a hard tip portion 10C provided at a distal end of the bending portion 10B. The bending portion 10B is configured to be freely bent by an operation of the operation part 11 (for example, an angle knob). The bending portion 10B can be bent in any direction and at any angle in accordance with, for example, a portion of a subject for which the scope 1 is used and can direct the tip portion 10C to a desired direction.

The interface adapter 4 connects the scope 1 and the portable information terminal 7 to each other. Specifically, the interface adapter 4, for example, connects with the scope 1 via the communication cable 13 to be able to communicate with the scope 1. In addition, the interface adapter 4 connects with the portable information terminal 7 in a wired manner or wirelessly to be able to communicate with the portable information terminal 7.

In addition, the interface adapter 4 receives from the scope 1 an imaging signal obtained by imaging the inside of a subject by an imaging sensor of the scope 1 and converts the received imaging signal into image data displayable by the portable information terminal 7. Then, the interface adapter 4 sends the converted image data to the portable information terminal.

The portable information terminal 7 is a general-purpose portable information terminal, such as a tablet terminal or a smartphone. The portable information terminal 7 has a display 7a that can display an image based on image data. The portable information terminal 7 receives from the interface adapter 4 a captured image and the like obtained by imaging the inside of a subject by the scope 1 and displays the received captured image by the display 7a. The display 7a has a display surface on which display pixels are arranged two-dimensionally, and pixel data that constitutes image data is drawn on the display pixels of the display surface to thereby display an image based on the image data. The portable information terminal 7 also functions as a user interface that controls the interface adapter 4.

### Interface Adapter 4

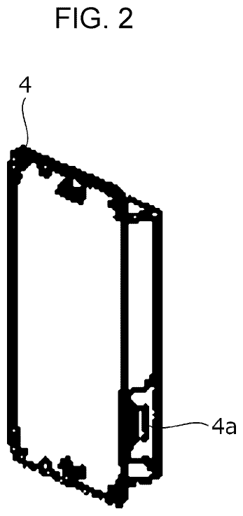

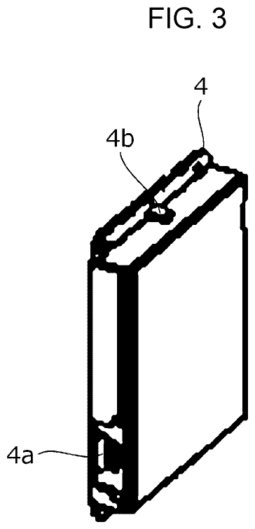

FIG. 2 and FIG. 3 each illustrate one example of the interface adapter 4. The direction in which the interface adapter 4 is viewed is different between FIG. 2 and FIG. 3. As illustrated in FIG. 2 and FIG. 3, the interface adapter 4 has a substantially rectangular parallelepiped shape and has a video input terminal 4a and a video output terminal 4b.

Each of the video input terminal 4a and the video output terminal 4b can be a terminal of various communication standards, such as a USB (Universal Serial Bus), a HDMI (High-Definition Multimedia Interface), a DVI (Digital Visual Interface), that can transmit video signals. HDMI is a registered trademark.

The video input terminal 4a is a terminal for connection with the scope 1 to be able to communicate with the scope 1. For example, the video input terminal 4a is a HDMI terminal, and the scope 1 is also provided with a HDMI terminal. The communication cable 13 is a HDMI cable, and the video input terminal 4a and the HDMI terminal of the scope 1 are connected to each other by the communication cable 13.

Connection between the interface adapter 4 and the scope 1 is, however, not limited to connection by the HDMI and may be wired connection by, other than the HDMI, a USB or a DVI or wireless connection by Bluetooth, near field communication, or the like. Bluetooth is a registered trademark. In addition, the interface adapter 4 and the scope 1 may be connected to each other via a conversion adapter that converts communication standards.

The video output terminal 4b is a terminal for connection with the portable information terminal 7 to be able to communicate with the portable information terminal 7. For example, the video output terminal 4b is a USB terminal, and the portable information terminal 7 is also provided with a USB terminal. Then, the video output terminal 4b and the USB terminal of the portable information terminal 7 are connected to each other by a USB cable.

Connection between the interface adapter 4 and the portable information terminal 7 is, however, not limited to connection by the USB and may be wired connection by, other than the USB, a HDMI or a DVI or wireless connection by Bluetooth, near field communication, or the like. In addition, the interface adapter 4 and the portable information terminal 7 may be connected to each other via a conversion adapter that converts communication standards.

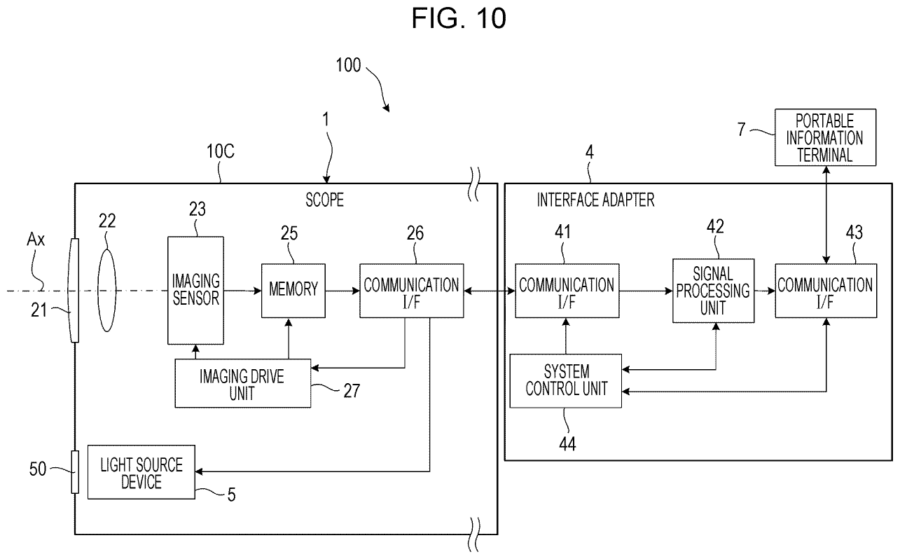

In addition, a circuit that controls the scope 1 and converts an imaging signal from the scope 1 into image data displayable by the portable information terminal 7 is provided inside the interface adapter 4. An internal configuration of the interface adapter 4 will be described later (for example, refer to FIG. 10).

State of Interface Adapter 4 and Portable Information Terminal 7 Before being Housed in Frame 8

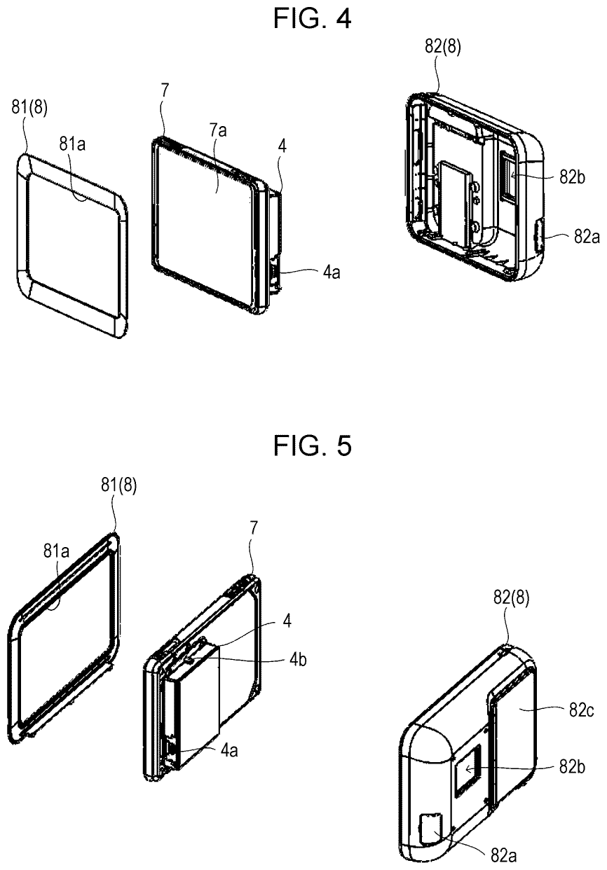

FIG. 4 and FIG. 5 each illustrate one example of a state of the interface adapter 4 and the portable information terminal 7 before being housed in the frame 8. The direction in which the interface adapter 4, the portable information terminal 7, and the frame 8 are viewed is different between FIG. 4 and FIG. 5. As illustrated in FIG. 4 and FIG. 5, the frame 8 includes a front cover 81 and a rear case 82.

The front cover 81 is a member that protects a front surface of the portable information terminal 7. The front cover 81 has an opening portion 81a for exposing the display 7a from the front cover 81 to enable display by the display 7a (touch panel) of the portable information terminal 7 and a touch operation with respect to the display 7a.

The rear case 82 is a member that protects a rear surface of the portable information terminal 7. The rear case 82 is provided with a cable insertion hole (not illustrated) that enables the communication cable 13 from the outside of the frame 8 to be connected to the video input terminal 4a of the interface adapter 4 housed in the frame 8. The rear case 82 is provided with a cable-insertion-hole cover 82a that closes the cable insertion hole. In addition, the rear case 82 is provided with a notation visual recognition hole 82b to enable a notation of standard conformity and the like on a housing of the interface adapter 4 to be visually recognized from the outside of the frame 8.

The interface adapter 4 is housed together with the portable information terminal 7 in the frame 8, and the interface adapter 4 is thereby fixed to the frame 8. In the example in FIG. 4 and FIG. 5, the interface adapter 4 is attached to the back surface of the portable information terminal 7 fixed by the frame 8 and is thereby fixed to the frame 8 via the portable information terminal 7.

The interface adapter 4 is attached to the portable information terminal 7 by, for example, screw fastening using a screw hole provided in the back surface of the portable information terminal 7. The interface adapter 4 may be attached to the portable information terminal 7 by a sheet metal or the like formed to correspond to the shape of the back surface of the interface adapter 4.

The interface adapter 4 is not limited to be fixed to the frame 8 via the portable information terminal 7. The interface adapter 4 may be attached directly to the frame 8. For example, the interface adapter 4 may be attached to the rear case 82 of the frame 8.

Further, a stand 82c may be provided on the outer side of the rear case 82. The stand 82c is provided on the rear case 82, for example, via a hinge, and a portion of the stand 82c is pulled from the rear case 82 to enable the frame 8 to stand on a horizontal place, such as a top board of a table.

In addition to the stand 82c or as an alternative to the stand 82c, a screw hole that can be used to attach the frame 8 to a wall-mounting metal fitting, an arm, a stand, or the like may be provided in the rear case 82. The screw hole can be a screw hole in accordance with, for example, VESA (Video Electronics Standards Association) standard or the like.

In the state in FIG. 4 and FIG. 5, the video output terminal 4b of the interface adapter 4 and the portable information terminal 7 are connected to each other via a communication cable (for example, a USB cable).

### State of Interface Adapter 4 and Portable Information Terminal 7 Attached to Rear Case 82

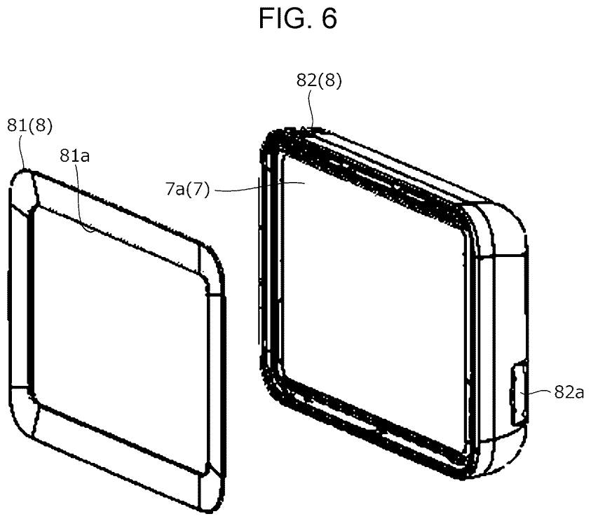

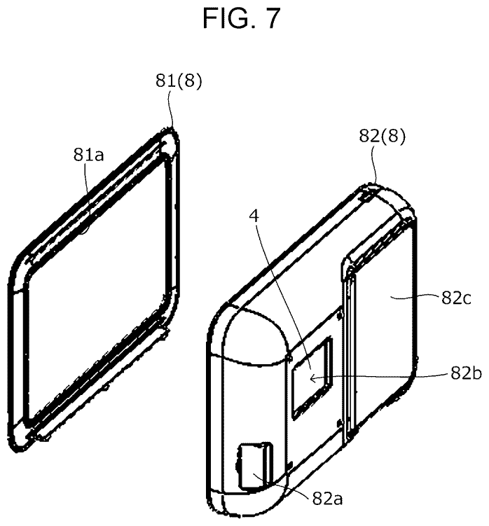

FIG. 6 and FIG. 7 each illustrate one example of a state of the interface adapter 4 and the portable information terminal 7 that are attached to the rear case 82. The direction in which the interface adapter 4, the portable information terminal 7, and the frame 8 are viewed is different between FIG. 6 and FIG. 7.

In a state in which, as illustrated in FIG. 4 and FIG. 5, the interface adapter 4 is attached to the back surface of the portable information terminal 7, the portable information terminal 7 and the interface adapter 4 are attached as illustrated in FIG. 6 and FIG. 7 to the inner side of the rear case 82. Consequently, a notation of standard conformity and the like on the housing of the interface adapter 4 are exposed from the notation visual recognition hole 82b. Removing the cable-insertion-hole cover 82a causes the video input terminal 4a of the interface adapter 4 to be in a state of being exposed from the cable insertion hole of the rear case 82.

### State of Front Cover 81 Attached to Rear Case 82

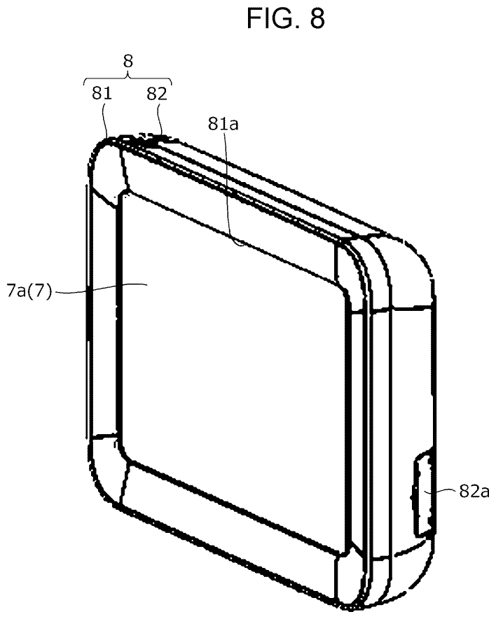

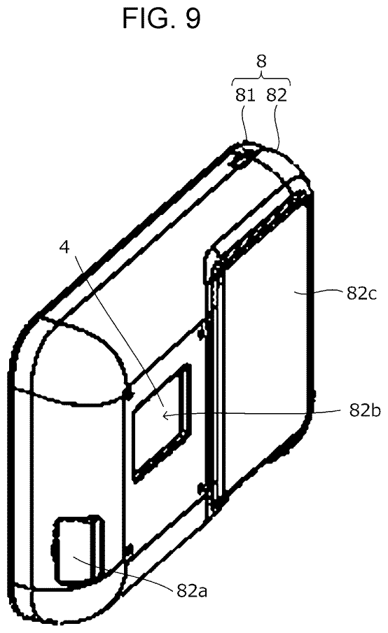

FIG. 8 and FIG. 9 each illustrate one example of a state of the front cover 81 that is attached to the rear case 82. The direction in which the frame 8 and the other members are viewed is different between FIG. 8 and FIG. 9.

In a state in which, as illustrated in FIG. 6 and FIG. 7, the portable information terminal 7 and the interface adapter 4 are attached to the rear case 82, the front cover 81 is attached as illustrated in FIG. 8 and FIG. 9 to the rear case 82. Consequently, the portable information terminal 7 and the interface adapter 4 are housed in the frame 8.

The frame 8 has a water-proof structure to suppress entrance of water, dust, and the like from the outside in the state illustrated in FIG. 8 and FIG. 9. For example, packing that fills a joint portion between the front cover 81 and the rear case 82 is provided between the front cover 81 and the rear case 82. In addition, a frame portion of the opening portion 81a of the front cover 81 is sealed to be in close contact with the display 7a of the portable information terminal 7, and the opening portion 81a is closed by the display 7a.

A frame portion of the notation visual recognition hole 82b of the rear case 82 is sealed to be in close with the housing of the interface adapter 4, and the notation visual recognition hole 82b is closed by the housing of the interface adapter 4.

A frame portion of the cable insertion hole of the rear case 82 is sealed to be in close contact with the housing of the interface adapter 4, and the cable insertion hole of the rear case 82 is closed by the housing of the interface adapter 4 when the cable-insertion-hole cover 82a is removed from the rear case 82. Further, an edge part of the cable insertion hole of the rear case 82 is sealed so that, when the cable-insertion-hole cover 82a is removed from the rear case 82, the cable insertion hole of the rear case 82 is closed by the communication cable 13 inserted through the cable insertion hole of the rear case 82.

In other words, the frame 8 has a water-proof structure such that the housed interface adapter 4 and the portable information terminal 7 are used to suppress entrance of water, dust, and the like to the inside of the frame 8. Consequently, even when the portable information terminal 7, the interface adapter 4, and the frame 8 are used in an outdoor environment or the like, entrance of water, dust, and the like to the inside of the frame 8 is suppressed, and the interface adapter 4 and the portable information terminal 7 can be protected.

As described above, the endoscope system 100 has a configuration in which the interface adapter 4 that converts an imaging signal obtained by an imaging sensor 23 of the scope 1 into image data displayable by the portable information terminal 7 is fixed to the frame 8 that houses the portable information terminal 7.

Consequently, processing of converting an imaging signal obtained by the imaging sensor 23 of the scope 1 into image data is performed by the interface adapter 4. Therefore, a general-purpose portable information terminal, such as a tablet terminal, can be used as the portable information terminal 7. This facilitates procurement, replacement, update, and the like of the portable information terminal 7.

Since the interface adapter 4 is fixed to the frame 8 that houses the portable information terminal 7, the portable information terminal 7 and the interface adapter 4 are integrated by the frame 8. This can facilitate handling for operating the scope 1 while observing with the portable information terminal 7 an image based on an imaging signal obtained by the imaging sensor 23 of the scope 1.

For example, a user (for example, a doctor or the like) of the endoscope system 100 can operate the scope 1 while observing an image without minding the position of the interface adapter 4 between the scope 1 and the portable information terminal 7. In addition, an accident in which, for example, the interface adapter 4 between the scope 1 and the portable information terminal 7 falls and pulls the scope 1 when a user operates the scope 1 while observing an image can be avoided.

Further, providing the interface adapter 4 between the scope 1 and the portable information terminal 7 eliminates the need to provide the scope 1 with an image processing circuit (for example, a signal processing unit 42) that converts an imaging signal obtained by the imaging sensor 23 of the scope 1 into image data. Thus, manufacturing costs of the scope 1 are reduced, and utilization of the scope 1 in single time use is facilitated.

### Internal Configurations of Scope 1 and Interface Adapter 4

FIG. 10 illustrates one example of internal configurations of the scope 1 and the interface adapter 4. As illustrated in FIG. 10, the tip portion 10C of the scope 1 is provided with an imaging optical system including an objective lens 21 and a lens group 22; the imaging sensor 23 that images a photographic subject through this imaging optical system; a memory 25, such as a RAM (Random Access Memory); a communication interface (FF) 26; an imaging drive unit 27; a light source device 5; and an illumination lens 50.

As the imaging sensor 23, a CCD (Charge Coupled Device) image sensor, a CMOS (Complementary Metal Oxide Semiconductor) image sensor, or the like is used. The imaging sensor 23 may perform image pick-up of a rolling shutter method or image pick-up of a global shutter method.

The imaging sensor 23 has a light receiving surface on which a plurality of pixels are disposed two-dimensionally and converts an optical image, which has been formed on the light receiving surface by the aforementioned imaging optical system, into an electrical signal (imaging signal) in each of the pixels. The imaging sensor 23 converts the converted imaging signal from an analogue signal into a digital signal of a predetermined bit number and outputs the imaging signal that has been converted into the digital signal to the memory 25. The imaging sensor 23 on which, for example, a color filter of a primary color, a complementary color, or the like is mounted is used. An aggregate of imaging signals output from the pixels of the light receiving surface of the imaging sensor 23 is referred to as a captured image signal.

A digital imaging signal output from the imaging sensor 23 is recorded temporarily in the memory 25. The communication interface (I/F) 26 is connected to a communication interface (I/F) 41 of the interface adapter 4. The communication interface 26 transmits the imaging signal recorded in the memory 25 to the interface adapter 4 through a signal wire in the communication cable 13.

The imaging drive unit 27 is connected to a system control unit 44 of the interface adapter 4 via the communication interface 26. The imaging drive unit 27 drives the imaging sensor 23 and the memory 25 on the basis of an instruction received by the communication interface 26 from the system control unit 44.

The light source device 5 can emit, as illumination light, normal light having an emission spectrum suitable for recognition by a human, such as a doctor. The normal light is, for example, white light or the like. Further, the light source device 5 may be able to emit, as illumination light, special light having an emission spectrum that is different from an emission spectrum of normal light and that is suitable for image analysis, such as IEE (Image-Enhanced Endoscopy: image emphasis observation), by a computer. For example, a semiconductor light source is used as a light source of the light source device 5.

The light source device 5 is connected to the system control unit 44 of the interface adapter 4 via the communication interface 26. The light source device 5 emits illumination light on the basis of an instruction received by the communication interface 26 from the system control unit 44.

The illumination lens 50 causes the illumination light emitted from the light source device 5 to be directed toward an object (for example, the inside of a subject) that is to be imaged by the imaging sensor 23 and the imaging optical system.

The interface adapter 4 includes the communication interface 41 connected to the communication interface 26 of the scope 1 by the communication cable 13; the signal processing unit 42; a communication interface (I/F) 43; and the system control unit 44.

The communication interface 41 has the video input terminal 4a illustrated in, for example, FIG. 2, FIG. 3, and the like. The communication interface 41 receives an imaging signal transmitted via the communication cable 13 from the communication interface 26 of the scope 1 and transfers the imaging signal to the signal processing unit 42.

The signal processing unit 42 incorporates a memory, such as a RAM, in which an imaging signal received from the communication interface 41 is temporarily recorded. The signal processing unit 42 subjects a captured image signal, which is an aggregate of imaging signals recorded in the memory, to processing (image processing, such as demosaic processing or gamma-correction processing) and generates image data in a form that can be displayed by the portable information terminal 7, which is a general-purpose terminal.

The communication interface 43 has the video output terminal 4b illustrated in, for example, FIG. 3 and the like and sends image data generated by the signal processing unit 42 to the portable information terminal 7.

The system control unit 44 controls each portion of the interface adapter 4 and also integrally controls the entirety of the endoscope system 100 by sending an instruction to the scope 1. For example, the system control unit 44 is one example of a controller of the imaging sensor 23, the controller controlling, via the imaging drive unit 27, imaging by the imaging sensor 23. The system control unit 44 is also one example of a controller of the light source device 5, the controller controlling irradiation with the illumination light by the light source device 5.

The system control unit 44 performs processing relating to brightness (exposure) of image data. For example, the system control unit 44 performs AE (Automatic Exposure) control in which, on the basis of image data generated by the signal processing unit 42, exposure of imaging by the imaging sensor 23 is automatically controlled to cause brightness of the image data to be brightness suitable for recognition by a human, such as a doctor or the like. The AE control is performed by automatic control of a diaphragm included in the imaging optical system, shutter speed of the imaging sensor 23, and ISO sensitivity.

The interface adapter 4 includes various types of processors that execute programs and perform processing, a RAM, and a ROM (Read Only Memory). The various types of the processors include a programmable logic device (PLD) that is a processor whose circuit configuration can be changed after manufacture, such as a CPU (Central Processing Unit) and a FPGA (Field Programmable Gate Array), which are general-purpose processors that execute programs and perform various types of processing, a dedicated electric circuit, such as an ASIC (Application Specific Integrated Circuit), which is a processor having a circuit configuration specially designed for executing specific processing, or the like. More specifically, structures of these various types of processors are electric circuits in which circuit elements, such as semiconductor elements, are combined together.

The interface adapter 4 may be constituted by one of various types of processors and may be constituted by a combination (for example, a combination of a plurality of FPGAs or a combination of a CPU and a FPGA) of two or more processors of the same type or different types.

### Hardware Configuration of Portable Information Terminal 7

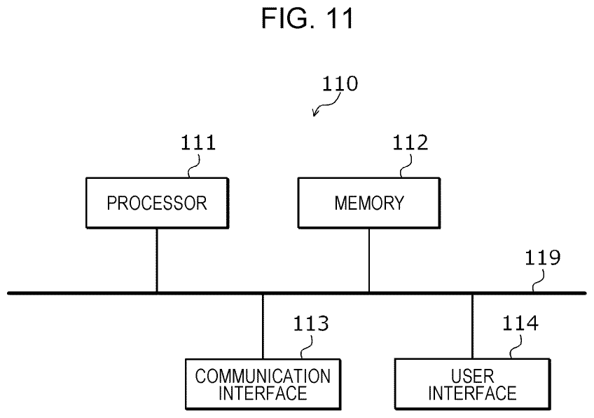

FIG. 11 illustrates one example of a hardware configuration of the portable information terminal 7. The portable information terminal 7 can be realized by, for example, a general-purpose information terminal 110 illustrated in FIG. 11. The information terminal 110 includes a processor 111, a memory 112, a communication interface 113, and a user interface 114. The processor 111, the memory 112, the communication interface 113, and the user interface 114 are connected to each other by, for example, a bus 119.

The processor 111 is a signal processing circuit and is, for example, a CPU (Central Processing Unit) in charge of controlling the entirety of the information terminal 110. The processor 111 may be realized by a different digital circuit, such as a FPGA (Field Programmable Gate Array) or a DSP (Digital Signal Processor). The processor 111 also may be realized by a combination of a plurality of digital circuits.

The memory 112 includes, for example, a main memory and an auxiliary memory. The main memory is, for example, a RAM. The main memory is used as a work area of the processor 111.

For example, the auxiliary memory is a nonvolatile memory, such as a magnetic disc, an optical disc, or a flash memory. The auxiliary memory stores various types of programs that operate the information terminal 110. The programs stored in the auxiliary memory are loaded into the main memory and executed by the processor 111.

The auxiliary memory may include a portable memory that can be detached from the information terminal 110. The portable memory is a USB flash drive, a memory card, such as a SD (Secure Digital) memory card, or the like.

The communication interface 113 is a communication interface that communicates with a portion (for example, the interface adapter 4) outside the information terminal 110. For example, the communication interface 113 is a wired communication interface having a terminal that can be connected via a communication cable to the video output terminal 4b of the interface adapter 4. Alternatively, the communication interface 113 may be a wireless communication interface that can wirelessly communicate with the interface adapter 4. The communication interface 113 is controlled by the processor 111.

The user interface 114 includes, for example, an input device that receives an operation input from a user, an output device that outputs information to a user, and the like. For example, the input device and the output device are realized by the display 7a configured as a touch panel. The user interface 114 may include, as the input device, a key, a remote controller, or the like. The user interface 114 may include, as the output device, a speaker, a vibrator, or the like. The user interface 114 is controlled by the processor 111.

For example, the touch panel included in the user interface 114 displays image data received via the communication interface 113 from the interface adapter 4. Consequently, an image based on an imaging signal obtained by the imaging sensor 23 of the scope 1 can be displayed with respect to a user, such as a doctor.

The touch panel included in the user interface 114 receives, through user operations, an instruction for imaging by the imaging sensor 23 of the scope 1 and an instruction for irradiation with illumination light by the light source device 5 of the scope 1. A control signal indicating a user operation received by the user interface 114 is sent to the system control unit 44 of the interface adapter 4 via the communication interface 113. On the basis of the received control signal, the system control unit 44 controls imaging by the imaging sensor 23 of the scope 1 and controls irradiation with the illumination light by the light source device 5 of the scope 1.

### Isolation Portion 45 Included in Interface Adapter 4

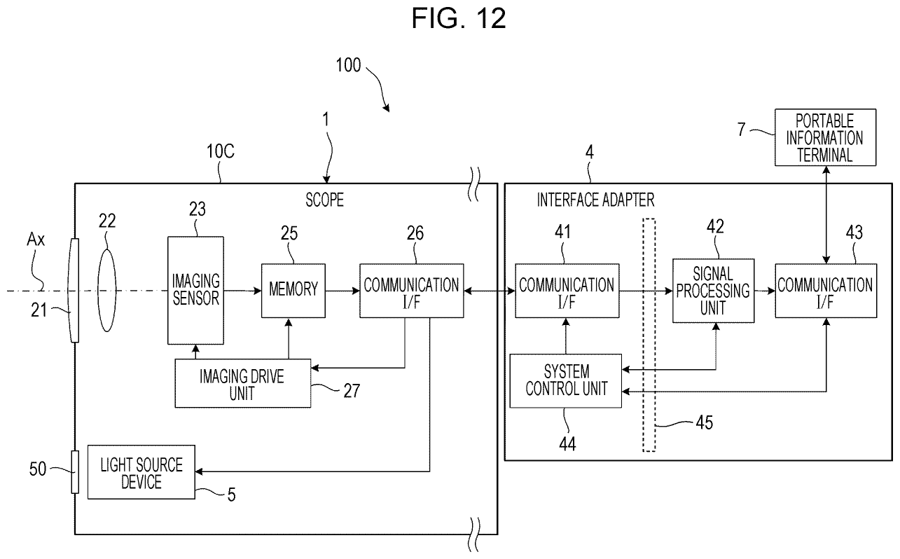

FIG. 12 illustrates one example of the isolation portion 45 included in the interface adapter 4. As illustrated in FIG. 12, the isolation portion 45 may be provided in the interface adapter 4. The isolation portion 45 electrically insulates a first circuit that is on the subject (patient) side and that is electrically connected to the scope 1 and a second circuit that is electrically connected to the portable information terminal 7 from each other. The first circuit includes, for example, the communication interface 41 and the system control unit 44. The second circuit includes, for example, the signal processing unit 42 and the communication interface 43.

The isolation portion 45 is constituted by an isolation device, such as a photocoupler or a pulse transformer. With the isolation portion 45, even when insulation failure occurs between the second circuit and a grounding portion (GND) due to a malfunction or the like, a situation in which unintended current flows from the second circuit to the first circuit on the subject side can be avoided, and security of the subject can be ensured.

### Power Supply Unit Included in Interface Adapter 4

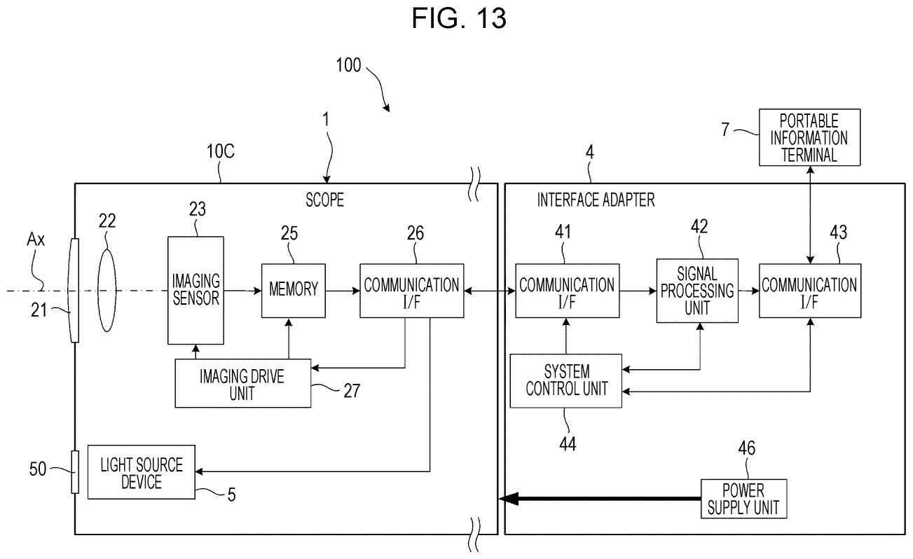

FIG. 13 illustrates one example of a power supply unit included in the interface adapter 4. As illustrated in FIG. 13, the interface adapter 4 includes the power supply unit 46. The power supply unit 46 supplies electric power to each portion of the interface adapter 4. The power supply unit 46 may be a unit that uses an external power source and may be a battery.

Further, the power supply unit 46 may supply electric power to the scope 1. For example, the power supply unit 46 supplies electric power to the scope 1 via a power supply line in the communication cable 13. Consequently, each portion of the scope 1 can be operated, without the power supply unit provided in the scope 1, by the electric power supplied by the power supply unit 46.

Power Supply Unit 46 Using Electric Power from Portable Information Terminal 7

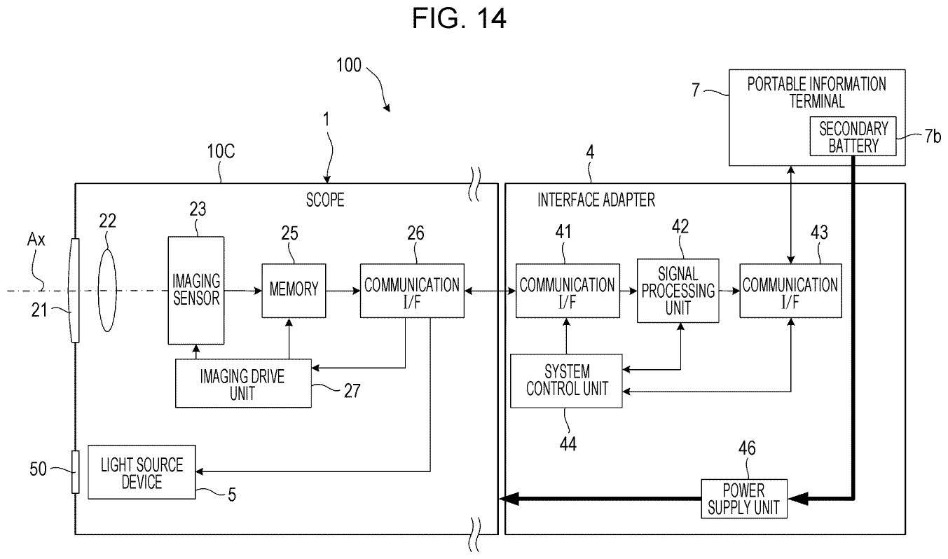

FIG. 14 illustrates one example of the power supply unit 46 that uses electric power from the portable information terminal 7. A secondary battery 7b is a secondary battery, such as a lithium ion battery included in the portable information terminal 7. The secondary battery 7b is used as a power source of the portable information terminal 7.

Further, the portable information terminal 7 may supply the electric power of the secondary battery 7b to the interface adapter 4. For example, the portable information terminal 7 and the interface adapter 4 are connected to each other via a communication cable, and the portable information terminal 7 supplies electric power to the interface adapter 4 via a power line in the communication cable. Alternatively, the portable information terminal 7 and the interface adapter 4 are connected to each other via a power supply cable, and the portable information terminal 7 may supply electric power to the interface adapter 4 via the power supply cable.

The power supply unit 46 of the interface adapter 4 operates using electric power supplied from the portable information terminal 7. Consequently, each portion of the interface adapter 4 can be operated, without a battery or the like provided in the interface adapter 4, using electric power supplied from the portable information terminal 7.

Further, each portion of the scope 1 can be operated by supplying to the scope 1 from the interface adapter 4 the electric power supplied from the portable information terminal 7. Consequently, the entirety of the endoscope system 100 can be operated using the secondary battery 7b of the portable information terminal 7. Charging management is thus facilitated. For example, a user of the endoscope system 100 can avoid lack of driving power of the scope 1 and the interface adapter 4 by only charging the secondary battery 7b of the portable information terminal 7.

As described above, the present specification discloses following matters.

(1)

An endoscope system including:
a scope that has an imaging sensor;
a frame that houses a portable information terminal; and
an interface adapter that converts an imaging signal obtained by the imaging sensor into image data displayable by the portable information terminal and sends the image data to the portable information terminal,
in which the interface adapter is fixed to the frame.

(2)

The endoscope system described in (1),
in which the interface adapter includes a controller of the imaging sensor.

(3)

The endoscope system described in (1) or (2),
in which the scope includes a light source device that generates illumination light for imaging by the imaging sensor, and
in which the interface adapter includes a controller of the light source device.

(4)

The endoscope system described in any one of (1) to (3),
in which the interface adapter performs processing relating to brightness of the image data.

(5)

The endoscope system described in any one of (1) to (4),
in which the interface adapter includes a first circuit, a second circuit, and an isolation portion that electrically insulates the first circuit and the second circuit from each other, the first circuit being electrically connected to the scope, the second circuit being electrically connected to the portable information terminal.

(6)

The endoscope system described in any one of (1) to (5),
in which the interface adapter supplies electric power to the scope.

(7)

The endoscope system described in (6),
in which the interface adapter supplies, to the scope, electric power that is supplied from the portable information terminal.

(8)

The endoscope system described in any one of (1) to (7),
in which the interface adapter is controlled by a signal from the portable information terminal.

(9)

An interface adapter of an endoscope system, the endoscope system including a scope that has an imaging sensor, and a frame that houses a portable information terminal having a display,
in which the interface adapter includes an image processor that converts an imaging signal obtained by the imaging sensor into image data displayable by the display and sends the image data to the portable information terminal, and the interface adapter is attachable to the frame.

While various embodiments have been described above with reference to the drawings, it is needless to say that the present invention is not limited to such examples. It is obvious that a person skilled in the art can conceive various modification examples and correction examples within the scope described in the claims. These examples are naturally understood to belong to the technical scope of the present invention. The components in the aforementioned embodiments may be combined together optionally within a range that does not deviate from the gist of the invention.

The present application is based on Japanese Patent Application (JP2021-134912), filed on Aug. 20, 2021, the contents of which are incorporated herein by reference.

### INDUSTRIAL APPLICABILITY

The present invention can provide an endoscope system and an interface adapter that can facilitate handling while enabling an image based on an imaging signal obtained by a scope to be displayed by a general-purpose portable information terminal.

### REFERENCE SIGNS LIST

1 scope
4 interface adapter
4a video input terminal
4b video output terminal
5 light source device
7 portable information terminal
7a display
7b secondary battery
8 frame
10 insertion part
10A soft part
10B bending portion
10C tip portion
11 operating unit
13 communication cable
22 lens group
23 imaging sensor
25, 112 memory
26, 41, 113 communication interface
27 imaging drive unit
42 signal processing unit
43 communication interface
44 system control unit
45 isolation portion
46 power supply unit
50 illumination lens
81 front cover
81a opening portion
82 rear case
82a cable-insertion-hole cover
82b notation visual recognition hole
82c stand
100 endoscope system
110 information terminal
111 processor
114 user interface
119 bus

What is claimed is:

1. An endoscope system comprising:
a scope that has an imaging sensor;
a frame that comprises a front cover and a rear case, and houses a portable information terminal in a space between the front cover and the rear case; and
an interface adapter that converts an imaging signal obtained by the imaging sensor into image data displayable by the portable information terminal and sends the image data to the portable information terminal,
wherein the interface adapter is fixed, via the portable information terminal, to the frame, in the space.

2. The endoscope system according to claim 1,
wherein the interface adapter comprises a controller of the imaging sensor.

3. The endoscope system according to claim 1,
wherein the scope comprises a light source device that generates illumination light for imaging by the imaging sensor, and
wherein the interface adapter comprises a controller of the light source device.

4. The endoscope system according to claim 2,
wherein the scope comprises a light source device that generates illumination light for imaging by the imaging sensor, and
wherein the interface adapter comprises a controller of the light source device.

5. The endoscope system according to claim 1,
wherein the interface adapter performs processing relating to brightness of the image data.

6. The endoscope system according to claim 2,
wherein the interface adapter performs processing relating to brightness of the image data.

7. The endoscope system according to claim 3,
wherein the interface adapter performs processing relating to brightness of the image data.

8. The endoscope system according to claim 1,
wherein the interface adapter comprises a first circuit, a second circuit, and an isolation portion that electrically insulates the first circuit and the second circuit from each other, the first circuit being electrically connected to the scope, the second circuit being electrically connected to the portable information terminal.

9. The endoscope system according to claim 2,
wherein the interface adapter comprises a first circuit, a second circuit, and an isolation portion that electrically insulates the first circuit and the second circuit from each other, the first circuit being electrically connected to the scope, the second circuit being electrically connected to the portable information terminal.

10. The endoscope system according to claim 3,
wherein the interface adapter comprises a first circuit, a second circuit, and an isolation portion that electrically insulates the first circuit and the second circuit from each other, the first circuit being electrically connected to the scope, the second circuit being electrically connected to the portable information terminal.

11. The endoscope system according to claim 1,
wherein the interface adapter supplies electric power to the scope.

12. The endoscope system according to claim 2,
wherein the interface adapter supplies electric power to the scope.

13. The endoscope system according to claim 3,
wherein the interface adapter supplies electric power to the scope.

14. The endoscope system according to claim 11,
wherein the interface adapter supplies, to the scope, electric power that is supplied from the portable information terminal.

15. The endoscope system according to claim 12,
wherein the interface adapter supplies, to the scope, electric power that is supplied from the portable information terminal.

16. The endoscope system according to claim 13,
wherein the interface adapter supplies, to the scope, electric power that is supplied from the portable information terminal.

17. The endoscope system according to claim 1,
wherein the interface adapter is controlled by a signal from the portable information terminal.

18. The endoscope system according to claim 2,
wherein the interface adapter is controlled by a signal from the portable information terminal.

19. The endoscope system according to claim 3,
wherein the interface adapter is controlled by a signal from the portable information terminal.

20. An interface adapter of an endoscope system, the endoscope system comprising a scope that comprises an imaging sensor, and a frame that houses a portable information terminal having a display,
wherein the interface adapter comprises an image processor that converts an imaging signal obtained by the imaging sensor into image data displayable by the display and sends the image data to the portable information terminal, and the interface adapter is attachable to the frame.
